(19) United States
(12) Patent Application Publication
(10) Pub. No.: US 2011/0178389 A1
(43) Pub. Date: Jul. 21, 2011

(54) FUSED IMAGE MOLDALITIES GUIDANCE

(75) Inventors: Dinesh Kumar, Rocklin, CA (US); Ramkrishnan Narayanan, Nevada City, CA (US)

(73) Assignee: EIGEN, INC., Grass Valley, CA (US)

(21) Appl. No.: 13/035,823

(22) Filed: Feb. 25, 2011

Related U.S. Application Data
(63) Continuation-in-part of application No. 12/434,990, filed on May 4, 2009.
(60) Provisional application No. 61/050,118, filed on May 2, 2008.
(60) Provisional application No. 61/148,521, filed on Jan. 30, 2009.

(51) Int. Cl. A61B 5/055; A61B 8/00

(52) U.S. Cl. 600/411

(57) ABSTRACT

An improved system and method (i.e. utility) for registration of medical images is provided. The utility registers a previously obtained volume (s) onto an ultrasound volume during an ultrasound procedure to produce a multimodal image. The multimodal image may be used to guide a medical procedure. In one arrangement, the multimodal image includes MRI information presented in the framework of a TRUS image during a TRUS procedure.

### CROSS REFERENCE TO RELATED APPLICATION

This application is a continuation-in-part of U.S. patent application Ser. No. 12/434,990, having a filing date of May 9, 2009 and which claims benefit of the filing date under 35 U.S.C. §119 to U.S. Provisional Application No. 61/050,118 entitled: “Fused Image Modalities Guidance” and having a filing date of May 2, 2008, and U.S. Provisional Application No. 61/148,521 entitled “Method for Fusion Guided Procedure” and having a filing date of Jan. 30, 2009, the entire contents of all of which are incorporated herein by reference.

### FIELD

The present disclosure pertains to the field of medical imaging, and more particularly to the registration of multiple medical images to allow for improved guidance of medical procedures. In one application, multiple medical images are coregistered into a multimodal image to aid urologists and other medical personnel in finding optimal target sites for biopsy and/or therapy.

### BACKGROUND

Medical imaging, including X-ray, magnetic resonance (MR), computed tomography (CT), ultrasound, and various combinations of these techniques are utilized to provide images of internal patient structure for diagnostic purposes as well as for interventional procedures. One application of medical imaging (e.g., 3-D imaging) is in the detection and/or treatment of prostate cancer. According to the National Cancer Institute (NCI), a man's chance of developing prostate cancer increases drastically from 1 in 10,000 before age 39 to 1 in 45 between 40 to59 and 1 in 7 after age 60. The overall probability of developing prostate cancer from birth to death is close to 1 in 6.

Traditionally either elevated Prostate Specific Antigen (PSA) level or Digital Rectal Examination (DRE) has been widely used as the standard for prostate cancer detection. For a physician to diagnose prostate cancer, a biopsy of the prostate must be performed. This is done on patients that have either high PSA levels or an irregular digital rectal exam (DRE), or on patients that have had previous negative biopsies but continue to have elevated PSA. Biopsy of the prostate requires that a number of tissue samples (i.e., cores) be obtained from various regions of the prostate. For instance, the prostate may be divided into six regions (i.e., sextant biopsy), apex, mid and base bilaterally, and one representative sample is randomly obtained from each sextant. Such random sampling continues to be the most commonly practiced method although it has received criticism in recent years on its inability to sample regions where there may be significant volumes of malignant tissues resulting in high false negative detection rates. Further using such random sampling it is estimated that the false negative rate is about 30% on the first biopsy. 3-D Transrectal Ultrasound (TRUS) guided prostate biopsy is a commonly used method to guide biopsy when testing for prostate cancer, mainly due to its ease of use and low cost.

Recently, it has been suggested that TRUS guidance may also be applicable for targeted focal therapy (TFT). In this regard, adoption of TFT for treatment of prostate cancer has been compared with the evolution of breast cancer treatment in women. Rather than perform a radical mastectomy, lumpectomy has become the treatment of choice for the majority of early-stage breast cancer cases. Likewise, some commentators believe that accurate targeting and ablation of cancerous prostate tissue (i.e., TFT) may eventually replace prostatectomy and/or whole gland treatment as the first choice for prostate treatment. Such targeted treatment has the potential to alleviate side effects of current treatment including, incontinence and/or impotence. Such commentators typically agree that the ability to visualize malignant or cancerous tissue during treatment will be of importance to achieve the accuracy of targeting necessary to achieve satisfactory results.

While TRUS provides a convenient platform for real-time guidance for either biopsy or therapy, it is believed that some malignant tissues can be isoechoic in TRUS. That is, differences between malignant cells and surrounding healthy tissue may not be discernable in the ultrasound image. Accordingly, using TRUS as a sole means of guidance may not allow for visually identifying potentially malignant tissue. Further, speckle and shadows make ultrasound images difficult to interpret, and many cancers are often undetected even after saturation biopsies that obtain several (>20) needle samples. Due to the difficulty of finding cancer, operators have often resorted to simply increasing the number of biopsy cores (e.g. saturation biopsy), which has been shown to offer no significant improvement in detection rate but instead increases morbidity. In order to alleviate this difficulty, a cancer atlas was proposed that provided a statistical probability image superposed on the patient's TRUS image to help pick locations that have been shown to harbor carcinoma, e.g. the peripheral zone constitutes about 80% of prostate cancer. While the use of a statistical map offers an improvement over the current standard of care, it is still limited in that it is estimated statistically from a large population of reconstructed and expert annotated 3-D histology specimen. That is, patient specific information is not available.

To improve the identification of potentially cancerous regions for biopsy or therapy procedures, it has been proposed to utilize different imaging modalities that may provide improved tissue contrast. Such different imaging modalities may allow for locating suspect regions or lesions within the prostate even when such regions/lesions are isoechoic. That is, imaging modalities like computed tomography (CT) and magnetic resonance imaging (MRI) can provide information that cannot be derived from TRUS imaging alone. While CT lacks good soft tissue contrast to help detect abnormalities within the prostate, it can be helpful in finding extra-capsular extensions when soft tissue extends to the periprostatic fat and adjacent structures, and seminal vesicle invasions.

MRI is generally considered to offer the best soft tissue contrast of all imaging modalities. Both anatomical (e.g., T₁, T₂) and functional MRI, e.g. dynamic contrast-enhanced (DCE), magnetic resonance spectroscopic imaging (MRSI) and diffusion-weighted imaging (DWI) can help visualize and quantify regions of the prostate based on specific attributes. Zonal structures within the gland cannot be visualized clearly on T1 images. However a hemorrhage can appear as high-signal intensity after a biopsy to distinguish normal and pathologic tissue. In T2 images, zone boundaries can be easily observed. Peripheral zone appears higher in intensity relative to the central and transition zone. Cancers in the peripheral zone are characterized by their lower signal intensity compared to neighboring regions.

DCE improves specificity over T₂ imaging in detecting cancer. It measures the vascularity of tissue based on the flow of blood and permeability of vessels. Tumors can be detected based on their early enhancement and early washout of the contrast agent. DWI measures the water diffusion in tissues. Increased cellular density in tumors reduces the signal intensity on apparent diffusion maps. MRSI is a four dimensional image that provides metabolite information at voxel locations. The relative concentrations of Choline, Citrate and Creatine help distinguish healthy tissue from tumors. Elevated Choline and Creatine levels and lowered citrate concentrations (ratio of choline to citrate) is a commonly used measure of malignancy.

Unfortunately, use of imaging modalities other than TRUS for biopsy and/or therapy typically provides a number of logistic problems. For instance, directly using MRI to navigate during biopsy or therapy can be complicated (e.g. requiring use of nonmagnetic materials) and expensive (e.g., MRI operating costs). This, need for specially designed tracking equipment, access to an MRI machine, and limited availability of machine time has resulted in very limited use of direct MRI-guided biopsy or therapy. CT imaging is likewise expensive and has limited access.

Accordingly, one solution is to register a pre-acquired image (e.g., an MRI or CT image), with a 3D TRUS image acquired during a procedure. Regions of interest identifiable in the pre-acquired image volume may be tied to corresponding locations within the TRUS image such that they may be visualized during/prior to biopsy target planning or therapeutic application. It is against this background that the present invention has been developed.

### SUMMARY

The term fusion is sometimes used to define the process of registering two images that are acquired via different imaging modalities. The present inventors have recognized that registration/fusion of images obtained from different modalities creates a number of complications. This is especially true in soft tissue applications where the shape of an object in two images may change between acquisitions of each image. Further, in the case of prostate imaging the frame of reference (FOR) of the acquired images is typically different. That is, multiple MRI volumes are obtained in high resolution transverse, coronal or sagittal planes respectively. These planes are usually in rough alignment with the patient's head-toe, anterior-posterior or left-right orientations. In contrast, TRUS images are often acquired while a patient lays on his side in a fetal position by reconstructing multiple rotated samples 2D frames to a 3D volume. The 2D image frames are obtained at various instances of rotation of the TRUS probe after insertion in to the rectal canal. The probe is inserted at an angle (approximately 30-45 degrees) to the patient's head-toe orientation. As a result the gland in MRI and TRUS will need to be rigidly aligned because their relative orientations are unknown at scan time. A further difficulty with these different modalities is that the intensity of objects in the images do not necessarily correspond. For instance, structures that appear bright in one modality (e.g., MRI) may appear dark in another modality (e.g., ultrasound). In addition, structures identified in one image (soft tissue in MRI) may be entirely absent in another image. Finally, the resolution of the images may also impact registration quality.

One aspect of the presented inventions is based upon the realization that, due to the FOR differences, image intensity differences between MRI and TRUS images, and/or the potential for the prostate to change shape between imaging by the MRI and TRUS scans, one of the few known correspondences between the prostate images is the boundary/surface model of the prostate. That is, the prostate is an elastic object that has a gland boundary or surface model that defines the volume of the prostate. In this regard, each point of the volume defined by the gland boundary of the prostate in one image should correspond to a point within a volume defined by a gland boundary of the prostate in the other image. Accordingly, it has been determined that registering the surface model of one of the images to the other image may provide an initial deformation that may then be applied to the field of the volume to be deformed. That is, elastic deformation of the image volume may occur based on an identified surface transformation between the boundaries.

According to a first aspect, a system and method (i.e., utility) is provided for use in medical imaging of a prostate of a patient. The utility includes obtaining a first 3D image volume from an MRI imaging device. Typically, this first 3D image volume is acquired from data storage. That is, the first 3D image volume is acquired at a time prior to a current procedure. A first shape or surface model may be obtained from the MRI image (e.g., a triangulated mesh describing the gland). The surface model can be manually or automatically extracted from all co-registered MRI image modalities. Any one of the MRI modalities is referred to as the first volume although it may usually be a T₂ volume), and all the remaining modalities are labeled complementary volumes. E.g. The first volume may be T₂ weighted MRI and the complementary volumes may comprise all other modalities not including T₂ like T₁, DCE (dynamic contrast-enhanced), DWI (diffusion weighted imaging), ADC (apparent diffusion coefficient) or other. The complementary volumes can typically be ones that help in the identification of suspicious regions but may not need to be necessarily visualized during biopsy. In the descriptions that follow, the first volume and all complementary volumes are assumed to be co-registered with each other as is usually the case. When a volume is referred to as the MRI volume, it refers collectively to the set of all co-registered volumes acquired from MRI (e.g. T₁, T₂, DCE, DWI, ADC, etc).

An ultrasound volume of the patient's prostate is then obtained, for example, through rotation of the TRUS probe, and the gland boundary is segmented in the ultrasound image. The ultrasound images acquired at various angular positions of the TRUS probe during rotation can be reconstructed to a rectangular grid uniformly through intensity interpolation to generate a 3D TRUS volume. The first volume is registered to the 3D TRUS volume, and a registered image of the 3D TRUS volume is generated in the same frame of reference (FOR) as the first volume (Alternately a registered image of the first volume may also be generated in the FOR of the ultrasound volume).

The registered image and the geometric transformation that relates the first volume with the ultrasound volume can be used to guide a medical procedure such as, for example, biopsy or brachytherapy. In one embodiment, the first volume data may be obtained from stored data. The first volume is usually a representative volume such as a T₂ weighted axial MRI. It is chosen because it is an anatomical volume where gland and zonal boundaries are clearly visible although occasionally T₁, DCE, DWI or a different volume may be considered the first volume. The utility may further include regions of interest identified prior to biopsy. These regions of interest are usually defined by a radiologist based on information available in MRI prior to biopsy, i.e. from T₁, T₂, DCE, DWI, MRSI or other volumes that can provide useful information about cancer. The regions of interest may be a few points, point clouds representing regions, or triangulated meshes.

In one aspect, segmenting the ultrasound volume to produce ultrasound surface model includes potentially using the first shape/surface model of the MRI to provide an initialized surface. This surface may be allowed to evolve in two or three dimensions. If the surface is processed on a slice-by-slice basis, vertices belonging to a first slice may provide initialization inputs to second vertices belonging to a second slice adjacent to the first slice and so on. Alternately, the vertices move in three dimensions simultaneously computing a 3D shape that describes the prostate.

According to another aspect, registering the first 3D volume to the ultrasound volume may include initially rigidly aligning the two volumes. The alignment may be based on heuristic information known from the MRI volume and the tracker information from the device. (The TRUS probe is attached to a tracking device that can determine the position of the probe in 3D). Additional rigid alignment input may also be provided by a user through specification of correspondences in both volumes.

According to another aspect, after rigid alignment, a surface correspondence between the first shape/surface model of the MRI image volume and the ultrasound image is established through surface registration. This may be the result of a nonrigid deformation applied to one of the surface models so as to align it with the other. According to yet another aspect, the deformation on the entire 3D rectangular grid (e.g., field deformation) can be estimated through elastically interpolating the geometry of the grid so as to preserve the boundary correspondences estimated from surface registration. Upon determining the field deformation, regions of interest in the MRI image may be transformed into the frame of reference of the ultrasound image.

According to another aspect, non-rigid intensity based registration may be used to find the deformation relating the two volumes with or without the aid of the segmented shapes.

According to another aspect, the intensity of one volume, say the reference, i.e. the first or the ultrasound can be determined in the frame of reference of the other through appropriate intensity interpolation after registration.

According to another aspect, a method is provided for use in imaging of a prostate of a patient. The method includes obtaining segmented MRI shape information for a prostate; extracting a derived ROI (regions of interest that may harbor cancer) from the MRI modalities; performing a transrectal ultrasound (TRUS) procedure on the prostate of the patient, wherein the segmented first shape information may be used to identify a three-dimensional TRUS surface model or the TRUS surface may be initialized and estimated independently from surface information from the first volume or first shape; surface registration to establish boundary correspondence between the two surface models; elastically warping one image to register it with the other based on the estimated boundary correspondence after surface registration; displaying the ROIs on a common FOR: first volume and warped 3D TRUS, or warped first volume and 3D TRUS); planning biopsy and/or therapy targets in the ROIs ; and guiding a medical procedure through navigation to these planned targets. This step may be performed on a slice-by-slice basis, may be done in two dimensions or in three dimensions, and/or may include generating a force field on a boundary of the segmented surface information; and propagating the force field through the derived volume to displace a plurality of voxels.

In accordance with another aspect, a system is provided for use in medical imaging of a prostate of a patient. The system may include a TRUS for obtaining a three-dimensional image of a prostate of a patient (3D TRUS); a storage device having stored there on the first volume and/or complementary volumes MRI; and a processor (e.g., a GPU) for registering the MRI volume to the 3D TRUS volumeof the prostate.

### BRIEF DESCRIPTION OF THE DRAWINGS

FIG. 1 shows a cross-sectional view of a trans-rectal ultrasound imaging system as applied to perform prostate imaging.

FIG. 2A illustrates a motorized scan of the TRUS of FIG. 1.

FIG. 2B illustrates two-dimensional images generated by the TRUS of FIG. 2A.

FIG. 2C illustrates a 3-D volume image generated from the two dimensional images of FIG. 2B.

FIG. 3 illustrates a user screen that provides four image panes.

FIG. 4 illustrates different images of a prostate acquired using different modalities.

FIG. 5 illustrates a side view of the images of FIG. 4.

FIGS. 6A-D illustrate a first prostate image, a second prostate image, overlaid prostate images prior to registration and overlaid prostate images after registration, respectively.

FIG. 7 illustrates fusing an MRI image with an ultrasound image to generate a multimodal image.

FIG. 8 illustrates a system for relating multimodality volumes, specifically here: MRI volume and 3D TRUS volume.

FIG. 9 illustrates a mesh surface model.

FIG. 10 illustrates the guide shape subsystem for segmentation of a 3D volume.

FIG. 11 illustrates the registration subsystem to relate all voxels in the 3D TRUS to the MRI volume.

FIG. 12 illustrates a surface deformation between images.

FIG. 13 illustrates a filed deformation between images.

### DETAILED DESCRIPTION

Reference will now be made to the accompanying drawings, which assist in illustrating the various pertinent features of the present disclosure. The following description is presented for purposes of illustration and description.

Disclosed herein are systems and methods that allow for registering images acquired from different imaging modalities (e.g., multimodal images) to a common frame of reference (FOR). In this regard, one or more images may be registered during, for example, an ultrasound guided procedure to provide enhanced patient information. Such registration of multimodal images is sometimes referred to as image fusion. In the application disclosed herein, a pre-acquired MRI image(s) of a prostate of a patient and a real-time TRUS image (e.g., 3D TRUS volume) of the prostate are registered such that information present in the MRI image(s) may be displayed in the FOR of the TRUS image to provide additional information that may be utilized for guiding a medical procedure on/at a desired location in the prostate. In the method disclosed for the purposes of illustration, a 3D TRUS volume is initially computed in the FOR of the MRI volume. That is, after registration of the 3D TRUS volume and MRI, the 3D TRUS volume is interpolated to the FOR of the MRI volume. The MRI volume may also be similarly computed in the FOR of TRUS in a similar manner (not described here).

### Overview

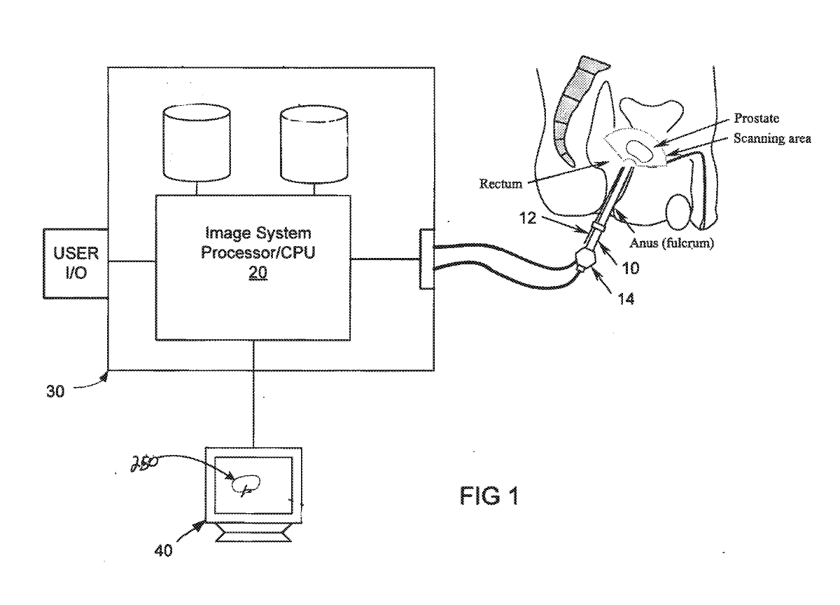

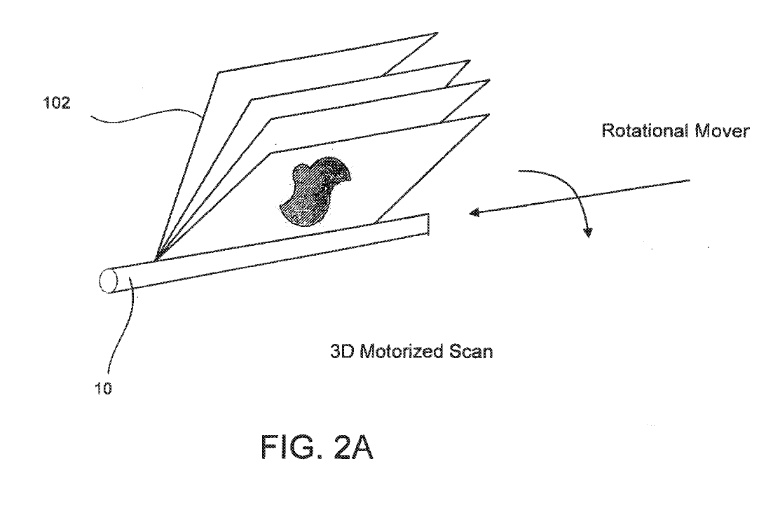

FIG. 1 illustrates a transrectal ultrasound (TRUS) imaging system that may be utilized to obtain a plurality of two-dimensional ultrasound images of a prostate 12. As shown, a TRUS probe 10 may be inserted rectally to scan an area of interest. In such an arrangement, a motor may sweep a transducer (not shown) of the ultrasound probe 10 over a radial area of interest. Accordingly, the probe 10 may acquire plurality of individual images while being rotated through the area of interest (See FIGS. 2A-C). Each of these individual images may be represented as a two-dimensional image. Initially, such images may be in a polar coordinate system. In such an instance, it may be beneficial for processing to resample these images into a rectangular coordinate system. In any case, the two-dimensional images may be combined to generate a three-dimensional image (See FIG. 2C).

A computer system 30 runs application software and computer programs which may control the TRUS system components, provide a user interface, monitor 40, and control various features of the imaging system. In the present embodiment, the monitor 40 is operative to display reconstructions of the prostate image 250. The computer system may also perform the multimodal image fusion functionality discussed herein. The software may be originally provided on computer-readable media, such as compact disks (CDs), magnetic tape, or other mass storage medium. Alternatively, the software may be downloaded from electronic links such as a host or vendor website. The software is installed onto the computer system hard drive and/or electronic memory, and is accessed and controlled by the computer's operating system. Software updates are also electronically available on mass storage media or downloadable from the host or vendor website. The software, as provided on the computer-readable media or downloaded from electronic links, represents a computer program product usable with a programmable computer processor having computer-readable program code embodied therein. The software contains one or more programming modules, subroutines, computer links, and compilations of executable code, which perform the functions of the imaging system. The user interacts with the software via keyboard, mouse, voice recognition, and other user-interface devices (e.g., user I/O devices) connected to the computer system.

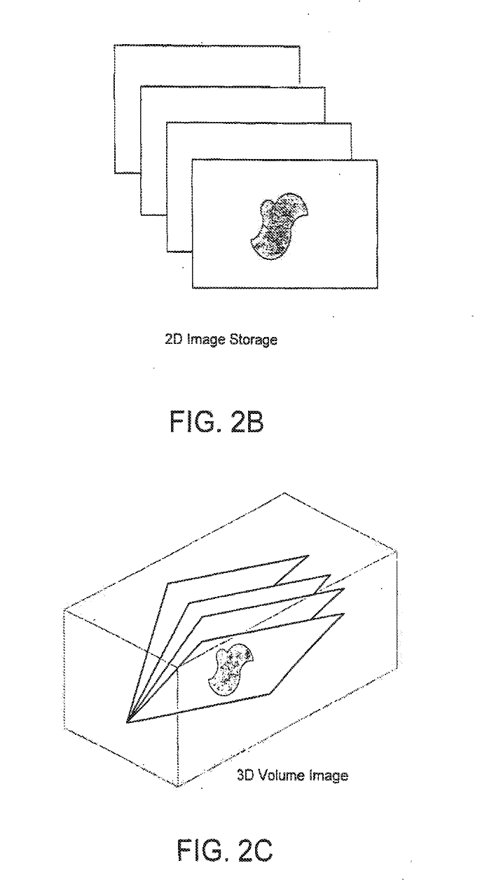

In order to generate an accurate surface model of the prostate from the 2D ultrasound images (e.g., image slices), the ultrasound images require segmentation. Segmentation refers to the process of partitioning a digital image into multiple segments (sets of pixels) with the goal of isolating an object of interest. As will be appreciated, ultrasound images often do not contain sharp boundaries between a structure of interest and background of the image. That is, while a structure, such as a prostate, may be visible within the image, the exact boundaries of the structure may be difficult to identify. This is illustrated in FIG. 3 in the bottom left panel. As shown, the prostate 250 in the ultrasound image 204 lacks clear boundaries. Accordingly, it is desirable to segment the images into a limited volume of interest (e.g., triangulated meshed surface model). Segmentation may be done manually or in an automated procedure. One method for segmenting a prostate is set forth in U.S. Pat. No. 7,804,989 the entire contents of which are incorporated herein. However, it will be appreciated that the present system is not limited to any particular segmentation system. Such segmentation systems and methods often generate boundary information slice by slice for an entire volume. As shown in the upper right panel 206 of FIG. 3. Once segmented, the boundary of the prostate 250 may be displayed on the prostate image.

Once the boundaries are determined, volumetric information may be obtained and/or a detailed 3D mesh surface model 254 may be created. See for instance the bottom right panel 208 of the display of FIG. 3. Such a 3D surface model may be utilized to, for example, guide biopsy or therapy. Further, the segmentation system and method may be implemented in ultrasound systems such that the detailed surface model may be generated while a TRUS probe remains positioned relative to the prostrate. That is, a surface model may be created in substantially real-time.

As shown in FIG. 1, the probe 10 includes a biopsy gun 8. Such a gun 8 may include a spring driven needle that is operated to obtain a core from desired area within the prostate. It will be appreciated that in therapy arrangements the biopsy gun may be absent and the imaging system may be operative to guide a therapy device (e.g. guide arm) that allows for targeting tissue within the prostate. In this regard, the TRUS volume may provide guidance for an introducer (e.g., needle, trocar etc.) of a targeted focal therapy (TFT) device. Such TFT devices typically ablate cancer foci within the prostate using any one of a number of ablative modalities. These modalities include, without limitation, cryotherapy, brachytherapy, targeted seed implantation, high-intensity focused ultrasound therapy (HIFU) and/or photodynamic therapy (PDT). In any of these focal therapy modalities, it may be necessary to accurately guide an introducer to desired foci within the prostate.

While TRUS is a relatively easy and low cost method of generating real-time images and identifying structures of interest, several shortcomings exist. For instance, some malignant cells and/or cancers may be isochoic. That is, the difference between malignant cells and healthy surrounding tissue may not be apparent or otherwise discernable in an ultrasound image. Further, speckle and shadows in ultrasound images may make images difficult to interpret. Stated otherwise, ultrasound may not, in some instances, provide detailed enough image information to identify tissue or regions of interest.

Other medical imaging modalities may provide significant clinical value, overcoming some of these difficulties. In particular, Magnetic Resonance Imaging (MRI) modalities may expose tissues or cancers that are isochoic in TRUS, and therefore indistinguishable from normal tissue in ultrasound imaging. As will be appreciated, MRI is a medical imaging technique used in radiology to visualize detailed internal structures. The good contrast it provides between different soft tissues of the body make it especially useful compared with other medical imaging techniques such as computed tomography (CT), X-rays or ultrasound. MRI uses a powerful magnetic field to align the magnetization of some atoms in the body, and then uses radio frequency fields to systematically alter the alignment of this magnetization. This information is recorded to construct an image of the scanned area of the body.

A typical MRI examination consists of a plurality of sequences, each of which is chosen to provide a particular type of information about the subject tissues. Stated otherwise, most MRI images include a plurality of different images/volumes (e.g., resulting from different applied signals) that are co-registered to the same frame of reference. When a volume is referred to as an MRI volume herein, it refers collectively to the set of all co-registered volumes acquired from MRI (e.g. T₁, T₂, DCE, DWI, ADC, etc). For example, the MRI volume may be T₂ weighted MRI and the complementary volumes may comprise all other modalities not including T₂ like T₁, DCE, DWI, ADC or other. The complementary volumes can typically be ones that help in the identification of suspicious regions but may not need to be necessarily visualized during biopsy or TFT. In the descriptions that follow, the first volume and all complementary volumes are assumed to be co-registered with each other as is usually the case.

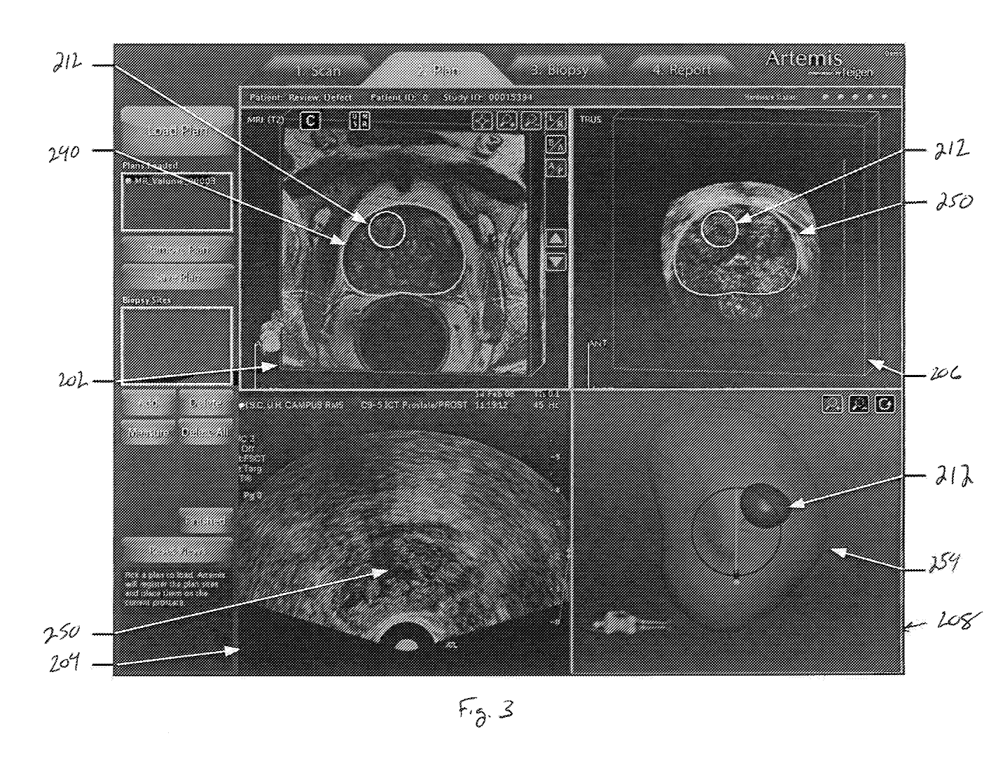

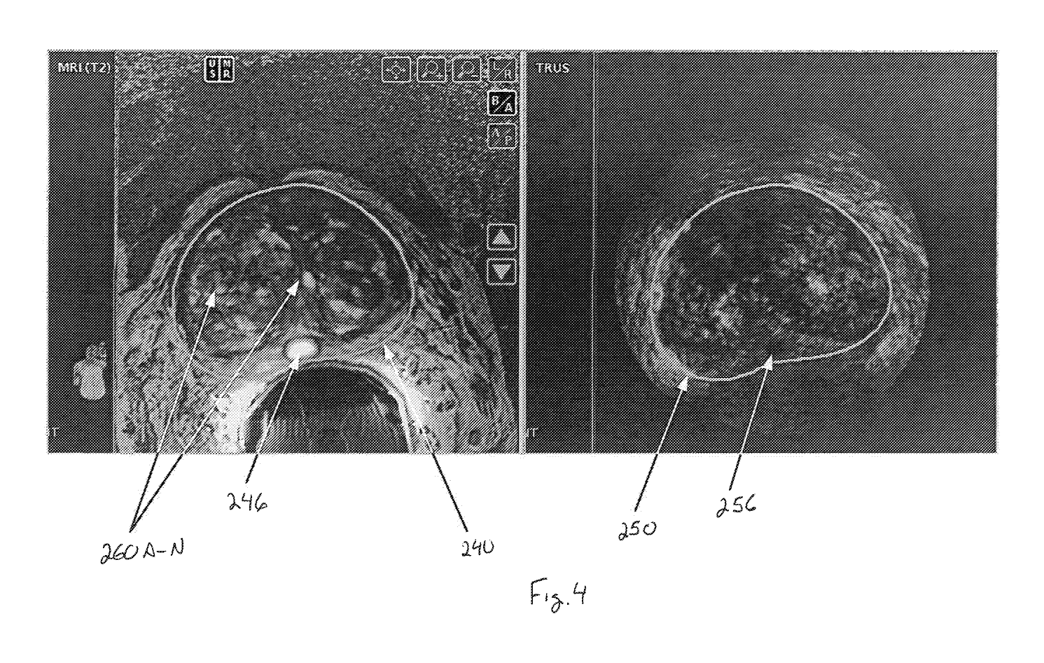

Scan times of MRI scanners can vary but typically requires at least a few minutes to acquire an image and some older models can require up to 40 minutes for the entire procedure. Accordingly, use of such MRI scanners for real-time guidance is limited. MRI scanners typically generate multiple two-dimensional cross-sections (slices) of tissue and these slices are stacked to produce three-dimensional reconstructions. That is, it is possible for a software program to build a volume by ‘stacking’ the individual slices one on top of the other. The program may then display the volume in an alternative manner. In this regard, MRI can generate cross-sectional images in any plane (including oblique planes). While the acquired in-plane resolution may be high, these cross-sectional images often have reduced clarity due to the thickness of the slices. For instance, the left panel of FIG. 4 illustrates a normal view (in-plane) of an MRI image plane. As can been seen, this image provides good resolution of structures of interest within the image. In contrast, the left panel of FIG. 5 illustrates an oblique plane that extends through multiple stacked MRI planes. As shown, the structures in these oblique views are difficult to discern due to the thick plane slices of the MRI. While it is possible to smooth such oblique images using smoothing algorithms, the contrast of structures in these slices may be reduced. In this regard, the soft tissue contrast that makes MRI desirable can be lost. Stated otherwise, most MRI images fail to produce data that can be reconstructed in any plane without loss of image quality.

Segmentation of MRI images is typically performed on a slice-by-slice basis by a radiologist. More specifically, a trained MRI operator manually tracks the boundaries of prostrate in multiple images slices or inputs initial points that allow a segmentation processor to identify the boundary. For instance, an operator may provide basic initialization inputs to the segmentation processor to generate an initial contour that is further processed by the processor to generate the segmented boundary. A typical initialization input could involve the selection of a few points that are non-coplanar along the boundary of the gland. The processor may operate on a single plane in the 3D MRI image, i.e. refining only points that lie on this plane. In some arrangements, the processor may operate directly in 3D using fully spatial information to allow points to move freely in three dimensions.

Typically, the 3D MRI image is divided into a number of slices, and the boundary of the gland is individually computed on each slice. That is, each slice is individually segmented, in parallel or in sequence. In some instances, the boundaries in one slice may be allowed to propagate across neighboring slices to provide a starting initialization for the neighboring slices. Once all slices are segmented, the volume of interest, when viewed from the side, may have a stair-step appearance. To provide a smooth surface model the system either incorporates a smoothing regularization within the segmentation framework or may apply a smoothing filter after segmentation using various algorithms on the volume (e.g. prostate). That is, the system is operative to utilize the stored boundaries to generate a 3D surface model and volume for the prostate of the MRI image.

Despite the advantages of using MRI to identify ROI within a prostate, ultrasound and TRUS in particular remains a more practical method for performing a biopsy or treatment procedure due to the cost, complexity and time constraints associated with direct MRI guided procedures. Thus, it has been recognized that it would be desirable to overlay or integrate information obtained from a pre-acquired MRI image with a real-time TRUS image to aid in selecting locations for biopsy or treatment as well as for guiding instruments during such procedures. In such an arrangement, the MRI and TRUS images may be registered, and the two registered volumes can be visualized simultaneously (e.g. side-by-side). Locations on MRI can be directly visually correlated with corresponding locations on TRUS, and the ROIs identified on MRI can also be displayed on TRUS.

Because the two images are obtained at different times, there may be a change in shape of the prostate related to its growth or shrinkage, patient movement or position, deformation of the prostate caused by the TRUS probe, peristalsis, abdominal contents, etc. Further, the images may be acquired from different perspectives relative to the patient. Accordingly, use of such a previously acquired MRI image with a current TRUS image will require registration of the images. For instance, these image volumes may need to be rigidly rotated to align with the images into a common frame of reference. Further, once the imaged are rigidly aligned, one of the images may need to be elastically deformed to match the other image.

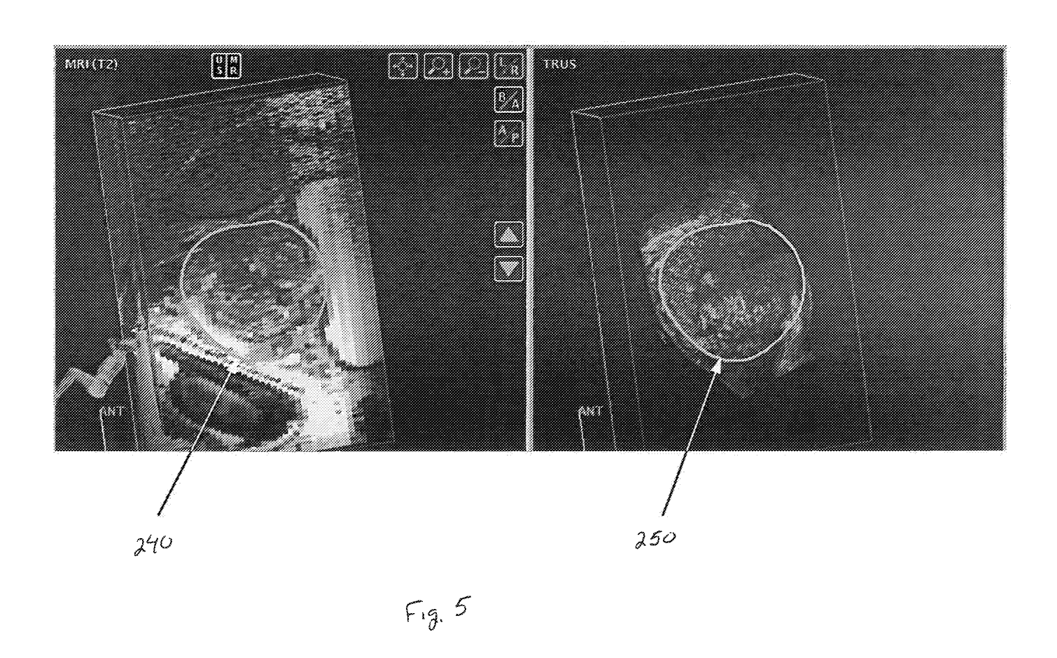

FIGS. 6A-D illustrate the need to register two volumes of a single prostate that were obtained using different imaging modalities by examining the shape differences between their respective surface models. Registration is used to find a deformation between similar anatomical objects such that a point-to-point correspondence is established between the images being registered. The correspondence means that position of similar tissues or structures is know in both images. FIGS. 6A and 6B illustrate first and second surface models 240 and 250, for example, as may be rendered on an output device of physician. These images may be from a common patient and may be obtained at first and second temporally distinct times and, in the present application, using different imaging modalities (e.g. TRUS and MRI). Though similar, the surface models 240, 250 are not aligned as shown by an exemplary overlay of the images prior to registration (e.g., rigid and/or elastic registration). See FIG. 6C. In order to effectively align the images 240, 250 to allow transfer of data (e.g., MRI) from a frame of reference of one of the images to a frame of reference of the other image, the images must be rigidly aligned to a common reference frame and then the one image (e.g., 240) may be deformed to match the shape of the other image (e.g., 250). In this regard, corresponding structures or landmarks of the images may be aligned to position the images in a common reference frame. See FIG. 6D. While simple in concept, the actual procedure is complicated by the use of different image modalities.

### Trus-Mri Registration/Fusion

The registration of different images into a common frame of reference can be performed in a number of different ways. When two images are acquired from a single imaging modality (e.g., two x-ray images, two ultrasound images etc), the two images typically include significant commonality. For instance, such images are often acquired from the same perspective and share a common frame of reference (e.g., sagittal, coronal etc.). Likewise, images acquired by a common modality will typically having matching or similar intensity relationships between corresponding features in respective images. That is, objects in the images (e.g., bone, soft tissue) will often have substantially similar brightness (e.g., on a grey scale). Accordingly, similar objects in these images may be utilized as fiduciary markers for aligning the images.

The term fusion is sometimes used to define the process of registering two images that are acquired via different imaging modalities. As noted above, different imaging modalities may provide different benefits. For instance, ultrasound provides an economical real-time imaging system while MRI can provide detailed tissue information that cannot be observed on ultrasound. However, the registration/fusion of these different modalities poses several challenges. This is especially true in soft tissue applications such as prostate imaging where the shape of an object in two images may change between acquisition of each image. Further, in the case of prostate imaging, the frame of reference (FOR) of the acquired images is typically different. That is, MRI prostate images may typically be roughly aligned with the patient positioning (head to toe, anterior to posterior and left to right). In contrast, TRUS images are often acquired while a patient lays on his side in a fetal position. Image acquisition is dependent on the angle of insertion of the probe introducing its own local reference (FOR). The result is that the images are initially 30-45 degrees out of alignment when the images are viewed in sagittal direction, and may be out of alignment in other directions as well by a several degrees. A further difficulty with these different modalities is that the intensity of objects in the images do not necessarily correspond. For instance, structures that appear bright in one modality (e.g., MRI) may be appear dark in another modality (e.g., ultrasound). Referring briefly to FIG. 4, it is noted that the urethra 246 of the MRI prostate image 240 set forth in the left hand panel is bright whereas the urethra 256 of the US prostate image 250 of the right hand panel is dark. In addition, structures of interest 260 A-N found in one image (soft tissue in MRI) may be entirely absent in the other image. Intensity based registration may increase computation times significantly compared to determining boundary correspondences. The slice thickness in MRI can be large (large inter-slice spacing >3 mm, in-plane resolution 0.5 mm) and presents challenges due to lack of information between slices to achieve high registration accuracy. Reconstruction of 3D TRUS on to the first volume results in interpolation of a high resolution image to the FOR of a low resolution image. The first volume is considered lower resolution due to its large slice thickness. (Displaying the first volume on 3D TRUS may appear very fuzzy because of the warping the thick slice planes). Simply stated, registering images obtained from different imaging modalities can be challenging.

One aspect of the presented inventions is based upon the realization that, due to the FOR differences and image intensity differences between MRI and TRUS prostate images, as well as the potential for the prostate to change shape between imaging by the MRI and TRUS devices, one of the only known correspondences between the prostate images from the different modalities is the boundary/surface of the prostate. That is, the prostate is an elastic object but has a gland boundary or surface that defines the volume of the prostate. In this regard, each point within the volume defined by the gland boundary in one image should correspond to a point within a volume defined by a gland boundary in the other image. Accordingly, it has been determined that registering the surface model of one of the images to the other image may provide an initial deformation that may then be applied to the field of the 3D volume to be deformed. That is, at the start of the TRUS procedure, the 3D TRUS volume is acquired from an ultrasound probe. This volume is segmented to extract the gland shape/surface model or boundary in the form of a surface. The method described here uses the shape information to identify corresponding features at the boundary of the prostate in the MRI image and 3D TRUS image followed by geometrically interpolating the displacement of individual voxels in the bulk/volume of the prostate image volume (within the shape) so as to align the two volumes. That is, a surface deformation (e.g. transformation) is initially identified between the two image volumes.

The surface transformation between these surface models is then used to drive the elastic deformation of points within the volume of the image. This elastic deformation with boundary correspondences has been found to provide a good approximation of the tissue movement within an elastic volume resulting from a change in shape of its outside surface. In this regard, the locations of objects of interest in the FOR of one volume may be accurately located in the FOR of the other volume. At the end of the registration, the registration parameters (parametric data such as knots, control points or a deformation field) are available, in addition to the 3D TRUS volume being registered to the MRI volume. Regions of interest (ROI) delineated on the MRI image or selected by a user from the MRI image may be exported to the FOR of the TRUS volume to guide biopsy planning or therapy. Both the first MRI volume (or any of the complementary volumes) and the registered 3D TRUS volume are visualized in various ways (slicing, panning, zooming, or rotating) side-by-side and blended with the ROI overlaid to provide additional guidance for biopsy planning or therapy. The user may plan biopsy targets by choosing regions within the ROI before proceeding to navigating to these targets.

Another aspect of the presented inventions is based upon the realization that interpolating the MRI volume in the FOR of TRUS for visualization maybe hard to visualize. The thick slices from MRI may make it fuzzy and hard to visualize after warping. That is, if the MRI image is deformed to fit the current real-time prostate image (e.g. sagittal plane), the MRI image may be viewed out of plane (e.g., See left pane FIG. 5) and in a manner where the resolution of the MRI image is compromised. For instance, if one of the points of interest 260A-N as illustrated in the MRI image of FIG. 4 is of interest, a user may not be able to identify this point of interest in an image as illustrated in the MRI image of FIG. 5. Accordingly, it has been determined that for MRI guidance purposes, it is desirable to transform the current or real-time TRUS image into the frame of reference of the MRI image. In this regard, points of interest may be identified in-plane of the MRI image (e.g., viewed in the plane having the best resolution) and such points of interest may then be transformed back into the current frame of reference of the TRUS prostate volume. For instance, referring to FIG. 3, the top left panel 202 illustrates the MRI-prostate image 240 and the top right panel 206 illustrates the registered TRUS image 250 (i.e., as registered to the MRI frame of reference). Accordingly, a region of interest 212 (e.g., as represented by the white circle) may be identified by user in the MRI image 240. Accordingly, this ROI may be illustrated in the registered TRUS image 250 and upon transformation using registration parameters this area of interest may be illustrated in the real-time 3D volume 254 as set forth in the bottom right panel 208. Accordingly, when disposed in the real-time frame of reference as illustrated in 3D volume 254, the region of interest 212 may be targeted for biopsy and/or ablation. In summary, it has been found that it is desirable to register the real-time image to the pre-acquired image to identify a transformation between the volumes. Upon identifying the transformation, such ROIs or areas of interest in the pre-acquired MRI image 240 (e.g., selected by a user or predefined regions of interest) may then be transformed into the frame of reference of the current real-time image 254. Accordingly, such areas of interest 212 may be displayed at their real-time location in the current image 254.

During a procedure, an operator may move through the MRI stack of images one by one to identify points of interest therein. Upon identifying each such point, the point may be saved by the system and identified in the frame of reference of the real-time image. Accordingly, the user may proceed through the entire stack of MRI images and select each point of interest within each image slice and subsequently target these points of interest. In a further arrangement, one or more points of interest or regions of interest may be pre-identified within the pre-acquired MRI image. As noted above, the MRI image is typically segmented prior to use in the system. In this regard, MRI images are typically segmented by a radiologist who is trained to read and identify objects within an MRI image. Accordingly, as the radiologist segments the outline of the prostate in each of the slices, the radiologist and/or an attendant physician may identify and outline regions of interest within one or more of the slices. For instance, as illustrated in FIG. 3, the region of interest 212 is illustrated as a circle in the normal view of the MRI image 240. Such a region of interest may extend across a number of adjacent planes of the MRI image and, similar to the surface of the prostate, may be smoothed to generate a boundary of a 3D region of interest as best illustrated by the spherical region of interest 212 in the surface model of TRUS illustrated in panel 208 of FIG. 3. Stated otherwise, one or more points or regions of interest may be predefined within the pre-acquired MRI image.

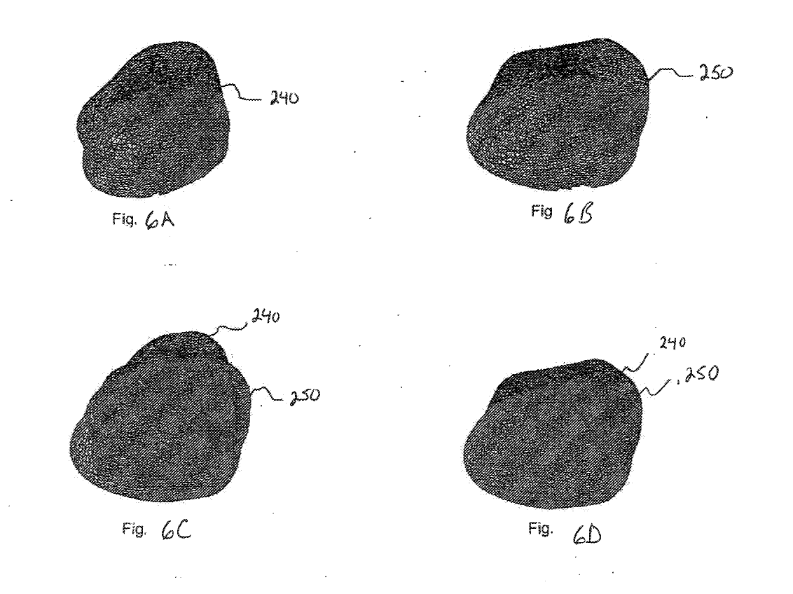

Once the MRI and TRUS images are registered in the MRI frame of reference, these images may be blended to create a composite image where information from both images is combined and displayed. This illustrated in FIG. 7 where in the middle panel, an image 280 is a 50% blend of each of the MRI image and the TRUS image. That is, each pixel within the resulting image may be a fifty percent blend of the corresponding pixel and the MRI image and the TRUS image. To improve the ability of users to select points of interest within the registered images, the present application further allows user adjustment of the combination or blend of images. In this regard, the user may adjust the blend between 100% of one volume (e.g., MRI volume) and 100% of the other volume (e.g., TRUS volume). As shown, the left hand panel 282 illustrates a 100% MRI image and the right hand panel 284 illustrates a 100% TRUS image. In this regard, a user may move back and forth between the images as represented in a common frame of references as a single image to see if there is correspondence between an object in the MRI volume and the TRUS volume.

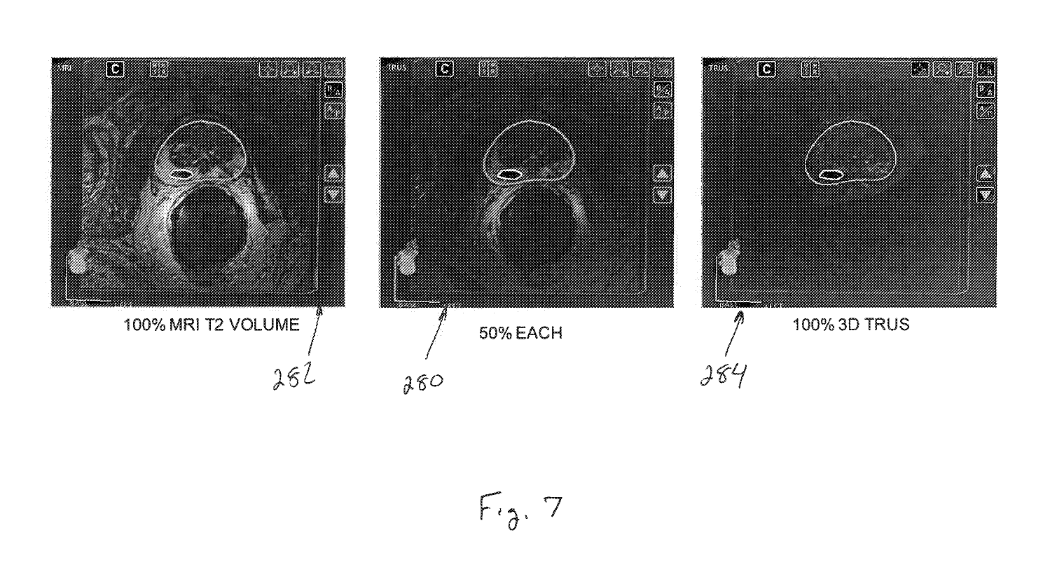

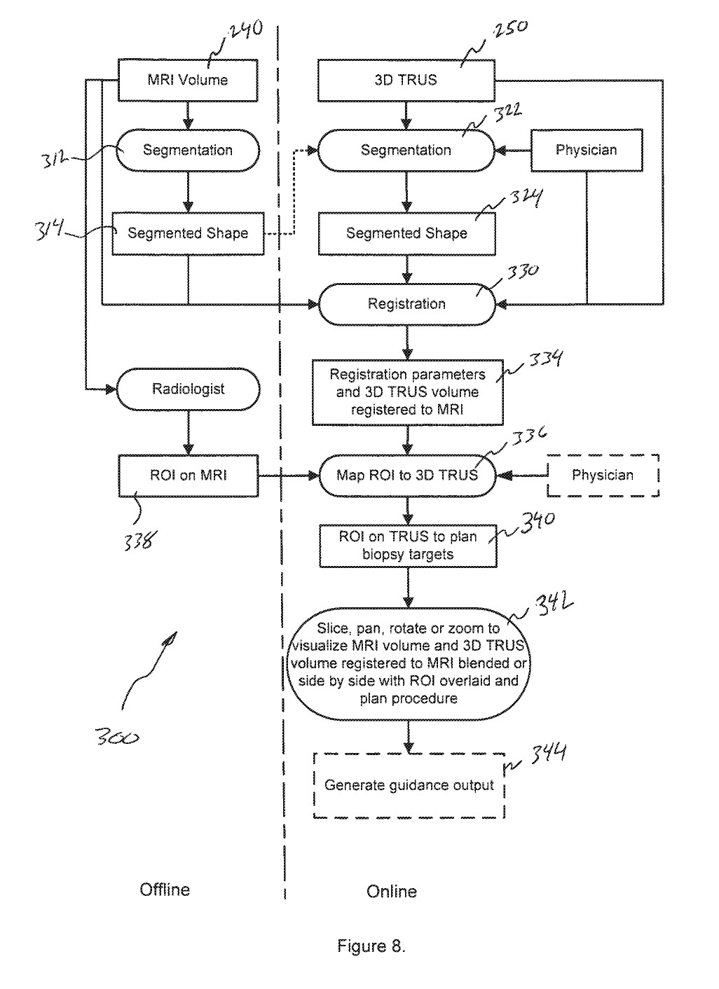

FIG. 8 illustrates an overall system 300 that provides multi-modal image fusion, which may be used in a biopsy and/or TFT application. As shown, the region to the left of the dotted line illustrates processing that can be done offline prior to biopsy or TFT. Initially, an MRI volume 310 (e.g., first volume and all complimentary volumes) is obtained and segmented 312 to provide a segmented shape or model surface 314, which in the present application may be represented in the form of a triangular mesh along the boundary of the prostate. An exemplary embodiment of such a mesh boundary 360 is provided in FIG. 9. It will be appreciated that each facet 362 of the triangulated mesh is defined by three vertices 364. Accordingly, the surface may be saved as a matrix of points (point list) followed by another matrix (face list) where each row specifies three vertices. Each vertex specified corresponds to a row number in the point list. For example a surface may contain the following two matrices in an ASCII file:

$$Point List = [ \begin{matrix}{x}_{1} & {y}_{1} & {z}_{1} \\ {x}_{2} & {y}_{2} & {z}_{2} \\ ⋮ & ⋮ & ⋮ \\ {x}_{n} & {y}_{n} & {z}_{n}\end{matrix} ] Face List = [ \begin{matrix}{v}_{i} & {v}_{j} & {v}_{k} \\ ⋮ & ⋮ & ⋮\end{matrix} ] \qquad eq . ( 1 )$$

The first row in the face list contains vi, vj and vk. This means the vertex in the ‘i’th row, ‘j’th row and ‘k’th row in the point list constitute one triangle. In addition to segmenting the MRI volume 310 in an offline procedure to generate a segmented shape/surface model 314, a radiologist can view the images in a suitable visualization environment and can identify regions of interest based on various characteristics observed in the MRI image, e.g., vascularity, diffusion, etc. Accordingly, in addition to the surface model, one or more regions or points of interest, which are also typically defined as a triangulated mesh or cloud of points, may be saved with the segmented surface 314. All of this data is made available at a common location during subsequent biopsy and/or therapy procedures. Such data may be available on CD/DVD, at a website, or via a network (LAN, WAN etc.).

To the right of the dotted line illustrated in FIG. 8 are steps performed during a guided procedure such as biopsy and/or targeted therapy. Initially, a 3D TRUS volume 320 is obtained. This volume 320 is segmented 322 automatically or by the direction of a physician 326 or other technician. This results in a segmented shape or surface 324 of the TRUS volume 320.

At this time, a surface model exists for both the MRI volume and the TRUS volume, where both surfaces represent the boundary of the patient's prostate. These surfaces 314, 324 are then registered 330 to identify a surface transformation between these shapes. This surface registration is then used to estimate a 3D field deformation for the current 3D TRUS volume 320 in order to identify the registration parameters 334 (e.g. field transformation) for the TRUS volume as registered to the MRI volume 334. At this time, the transformation between the TRUS volume 320 and the MRI volume 310 is completed and one of these volumes may be disposed in (e.g. transformed) frame of reference of the other volume, for instance, as set forth in FIG. 3 and FIG. 4. Accordingly, at this time the physician may identify points of interest 260A-N in the MRI image volume 310 and have those points of interest mapped to the 3D TRUS volume 320. That is, the application allows for the real-time selection of points in the MRI image volume and/or the registered ultrasound image. Further, such user selected points may be transformed and identified in their actual location in the current real-time 3D volume 320. Referring to FIG. 3, in such an instance a physician may identify a point in the MRI image 240 and this point may be identified in the registered TRUS image as well as in the real-time TRUS volume 254 illustrated in the bottom right pane of FIG. 3. In this regard, the ability to identify a point in the MRI and have this point displayed at its current real-time location allows a user to guide an instrument to such a location.

In an alternate arrangement, instead of the physician who is performing the real-time procedure selecting regions of interest from the MRI, such regions of interest 338 on the MRI image volume may be previously identified by a radiologist, (e.g., offline prior to the real-time procedure) and stored. In such a case, once the field transformation between the volumes is computed such a transformation may be applied to the pre-stored regions of interest 338 of the MRI data and these regions of interest may be mapped 336 to the 3D TRUS image 320. Again, this is illustrated in FIG. 3 where a circular region of interest 212 that is pre-stored within the MRI image of the top left panel is mapped to corresponding locations in the registered ultrasound image as well as the real-time ultrasound volume.

In any case, after mapping 336 regions of interest to the 3D TRUS volume, these regions of interest are displayed on the TRUS volume 320 such that a user may identify these regions of interest in a current real-time image or reconstructed volume for targeting 340. In addition, the system allows the user to manipulate 342 any of the images. In this regard, a user may slice, pan rotate zoom any or all of the 3D volumes. This includes the MRI volume, the registered TRUS volume and the real-time TRUS volume. Further, the user may variably blend the different images (e.g., see FIG. 7). Stated otherwise, a user may manipulate 342 volumes in order to identify points of interest therein. In a further arrangement, upon identifying a point of interest in the real-time image, a system may generate control outputs 344. Such control outputs may include providing target information (e.g., crosshairs) on the real-time image that allows for guiding a biopsy needle to an ROI or point of interest within the image. Alternatively, such outputs may include control outputs that operate, for example, an arm that guides an introducer to an ROI or point of interest within the image. Such guidance may be automated or semi-automated where a user has to finally introduce a trocar through tissue upon a guidance arm being properly aligned. At such time, one or more different TFT devices may be utilized to ablate tissue within the prostate.

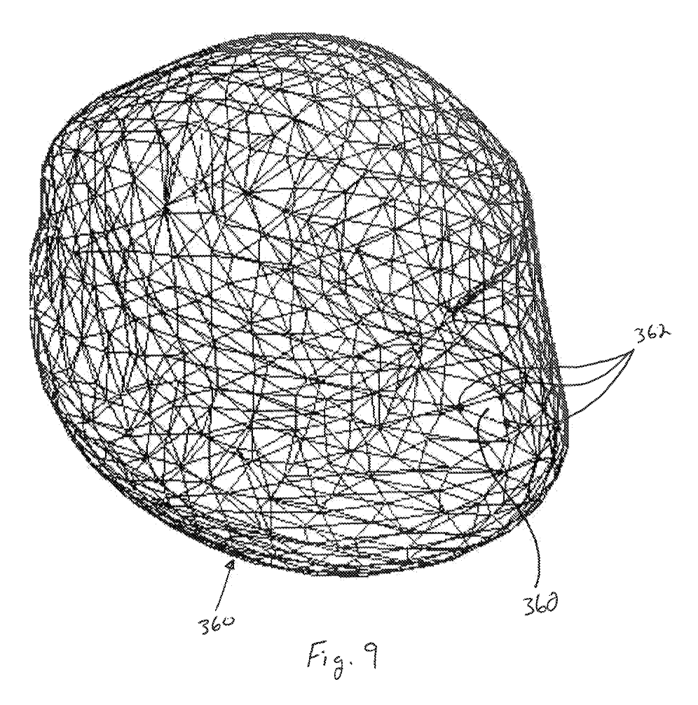

FIG. 10 shows a more detailed view of the segmentation performed on both the MRI image and 3D TRUS image. The procedure for segmenting these surfaces is similar and the following discussion applies to segmentation of both the MRI image and TRUS image, though discussed primarily in relation to the TRUS image. Further, it will be appreciated that various different algorithms may be used to implement segmentation (e.g., guide a shape processor and a morphing processor). FIG. 10 shows the segmentation of a 3D volume 410 such as a 3D TRUS volume or other volume through a basic surface initialization 412 provided by a physician or other operator 414. This initialization 412 may include the manual selection of a number of points (e.g., four) on the boundary of the gland in one or multiple dimensions (e.g., in first and second transverse planes) after which the system may interconnect these points to provide an initial shape 416. The initial shape 416 is iteratively updated by deforming processor 418 based on various factors or registration parameters like image gradient and shape smoothness to obtain a final shape 420 that hugs the boundary of the gland on the TRUS volume. The registration parameters can be, without limitation, the specific parameterization method, smoothness constraint or maximum number of iterations allowed etc. After segmentation, the operator may refine or edit 422 the surface by dynamically editing the shape (e.g. triangulated mesh) by providing one or more point inputs through point clicks on the sagittal, transverse or coronal views. If necessary, the process may then be repeated. In some instances it may be possible to use the shape/surface model from pre-acquired MRI. The initial shape is similarly iterated to obtain a final segmented shape from the 3D TRUS volume. Segmentation of the MRI volume may be done in a similar manner.

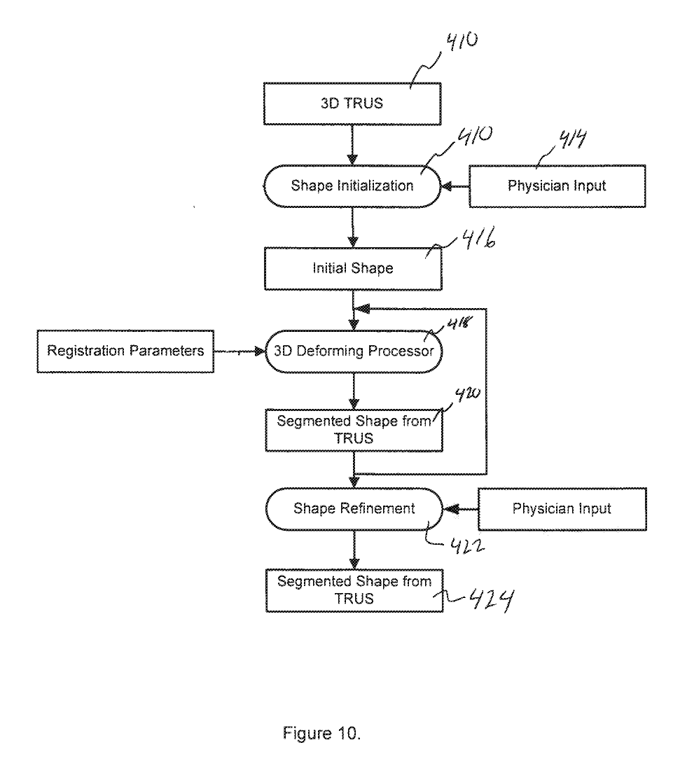

FIG. 11 illustrates the registration process. In this implementation both volumes 310, 312 are provided as input, with their respective surface shapes 314, 324 (e.g., triangulated mesh shapes). Specifically, the volumes are provided to a rigid alignment processor 440. An initial rigid alignment is applied to one of the volumes based on heuristics in addition to a user specified correspondence. That is, an initial rigid transformation is applied to one of the two volumes based on heuristics such as the tracker encoder values that localize the position of anatomies on the images in 3D space for the ultrasound volume 320. Additional alignment information may also be determined from the DICOM headers of the MRI volume which give image position and orientation information with respect to the patient. The MRI volume and 3D TRUS volume may be displayed after this initial alignment side-by-side. Upon further analysis, if the rigid orientations do not appear satisfactory, the physician may provide two or more points to orient the two volumes. For instance, the physician may identify common landmarks (e.g., urethra) in each image. Providing two or three points on corresponding planes rotates the entire volume about the normal to the plane based on the computed in-plane rotation estimated from a linear least squares fit. Providing four or more non-coplanar points will allow the simultaneous estimation of all 3D rigid parameters (three rotations and three translations). The physician has the ability to iteratively improve rigid alignment by specifying new corresponding fiducials on the previously aligned volumes. Additionally, the software can also allows the ability to go back to the previously specified alignment (undo), or revert to the original state, i.e. initial heuristic based alignment. When alignment is satisfactory, the rigid parameters 442 are saved to a file in a database, and the software allows the physician to proceed to non-rigid alignment.

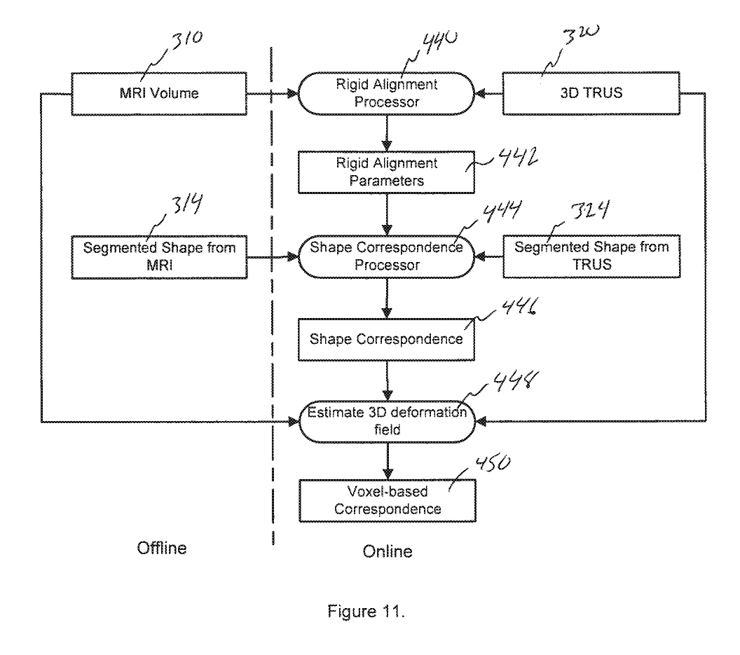

The rigid alignment parameters 442 are utilized by a shape correspondence processor 444 in conjunction with the segmented shapes 314, 324 to estimate correspondence along the boundary of the gland in MRI and 3D TRUS. This boundary or shape correspondence 446 is provided as input to a geometric interpolation—an elastic partial different equation used to model voxel position that may smoothly interpolate the deformation of the voxels within one of the volumes (deformation field) while preserving the boundary correspondence. Stated otherwise, the shape correspondence defines a surface transformation from one surface model (e.g., TRUS) to the other (e.g., MRI) and this surface transformation may then be used to calculate a 3D deformation field 448 for the image volume. Generally, the surface deformation may be applied through the volume using, for example, a Radial basis function or other parametric methods. Other implementations may include direct 3D intensity based registration where the bulk (voxels inside and outside the gland) may direct drive registration. Intensity based methods may also use shape information if available to improve performance. The correspondence between shapes (surface transformation) is computed as the displacement of vertices 370 from one surface so as to map to corresponding regions in the other surface. See FIG. 12. A suitable smooth parameterization is chosen to achieve this shape deformation. Without loss of generality one of the surfaces is called the model 314 (surface model from the MRI volume), and the other surface is called the target (surface model from 3D TRUS). The vertices from the model 314 are warped iteratively so as to relocate to the boundary of the target. At the end of the surface registration, a correspondence is achieved on the boundary. This correspondence is expressed as the joint pairs of vertices 370 on the model 314 and the vertices on the model after iteratively warping to match the target 324.

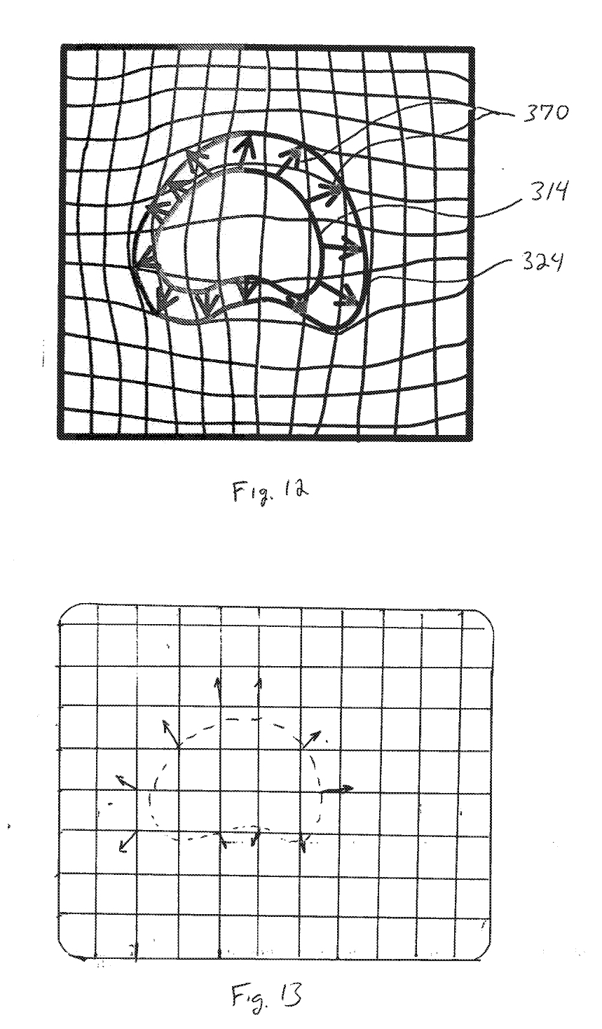

Stated otherwise, direction and displacement between corresponding vertices is identified. In this regard, displacement vectors are identified between the surfaces. Accordingly, these displacement vectors may be iteratively applied through voxels within a three-dimensional space of one of the images to elastically deform the interior of that image to the new boundary. FIG. 13 (not to scale), represents a two-dimensional array of voxels for purposes of illustration, but it will be appreciated that in practice represents a three-dimensional volume. As noted, the deformation vectors are known for each vertices of the surface. To deform the volume, these flexion vectors need to be carried through the interior of the volume. In this regard, each vector may be applied to the nearest grid point (e.g., pixel) in relation to the vertices of the surface. That is, the surface is disposed within the frame of reference of the three-dimensional volume and the vectors are applied to the nearest corresponding voxels. Once all of the vectors are applied to their nearest grid point, the volume is deformed (i.e., in accordance with predetermined elastic constraints) and the resulting surface is smoothed. Likewise, the new resulting vectors are applied to the next inner set of voxels and the process is repeated iteratively until volume is deformed through its interior. It has been determined that this type of deformation provides a good match to actual deformations applied to elastic objects.

An advantage of the techniques described in this implementation is their scalability with processor optimization (e.g., graphical processing unit (GPU) improvements). Images or surfaces can be split into several thousands of threads each executing independently. Data cooperation between threads is also made possible by the use of a shared memory. A GPU-compatible application programming language (API), e.g. nVidia's CUDA can be used to accomplish this task. It is generally preferable to design code that scales well with improving hardware to maximize resource usage. First the code is analyzed to see if data parallelization is possible. Otherwise algorithmic changes are suitably made so as bring about parallelization, again if this can be done. If parallelization is deemed feasible, the appropriate parameters on the GPU are set so as to maximize multiprocessor resource usage. This is done by finding the smallest data parallel thread, e.g. for vector addition, each vector component can be treated as an independent thread. This is followed by estimating the total number of threads required for the operation, and picking the appropriate thread block size that runs on each multiprocessor. For example, in CUDA selecting the size of each thread block that runs on a single multiprocessor determines the number of registers available for each thread, and the overall occupancy that can affect computation time. Other enhancements may involve, for example, coalescing memory addressing, avoiding bank conflicts, or minimizing device memory usage to further improve speed.

A strategy for GPU optimization for the processing steps is now described. First, segmentation of a prostate from MRI or segmentation of the prostate from TRUS guided by MRI may include allowing an initial surface to evolve so as to converge to the boundary of the respective volumes. Segmentation of the MRI may be performed in two or three dimensions. In either case, points intended to describe the prostate boundary evolve to boundary locations, e.g. locations with high gradients, or other criteria. Each vertex may be treated as a single thread so that it evolves to a location with high intensity gradient. At the same time, status of neighboring vertices for each vertex can also be maintained during the evolution to adhere to certain regularization criteria required to provide smooth surfaces.

Registration of a surface models of the gland from MRI and TRUS may include estimating surface correspondences, if not already available, to determine anatomical correspondence along the prostate boundaries from both modalities. This may be accomplished by a surface registration method using two vertex sets, for example sets A and B belonging to MRI and TRUS, respectively or vice versa. For each vertex in A, the nearest neighbor in B is found, and vice versa, to estimate the force and reverse forces acting on the respective vertices to match the corresponding set of vertices. The computations may be parallelized by allowing individual forces (forward and reverse) on each vertex to be computed independently. The forward force computations are parallelized by creating as many threads as there are vertices in A, and performing a nearest neighbor search. For example, a surface A having 1297 vertices could run as 40 threads/block containing 33 blocks. The threads corresponding to vertices beyond 1297 would not run any tasks. A similar procedure may be applied to compute the reverse force, i.e from B to A. Once forces are estimated, smoothness criteria may be similarly enforced as described in the segmentation step by maintaining the status of neighboring vertices for each vertex.

Finally, geometric interpolation satisfying the elastic partial differential equation (PDE) is solved to estimate the displacement of voxels from the MRI volume to 3D TRUS. This implicitly provides smoothness of the displacements while still satisfying boundary conditions. To compute the geometric deformation on the grid containing the MRI volume, it may be subdivided into numerous sub-blocks where voxels within each sub-block can query the positions of the neighboring voxels to estimate the finite difference approximations for the first and second degree derivatives of the elastic PDE. Each of the sub-blocks can be designed to run on a multiprocessor on the GPU. The interpolation may be performed iteratively using Jacobi parallel relaxation, wherein node positions for all nodes in the 3-D volume are updated after each iteration.

To summarize: there are two outputs from the fusion step. The first output is the 3D TRUS volume that is warped to align with the MRI volume. The volumes are visualized in various slice sections and orientations side-by-side or blended with the ROIs overlaid to plan targets for biopsy or therapy. The second output is the ROI that is mapped to the 3D TRUS volume from its definition on the MRI volume. This enables the display of the ROI overlay when it intersections with any slice section viewed on ultrasound during navigation while performing biopsy or therapy. The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A method for use in prostate treatment procedures where a pre-procedure Magnetic Resonance Imaging (MRI) image is utilized in conjunction with a current ultrasound image to guide a medical procedure, comprising:
obtaining, at a processing platform, a pre-acquired first three-dimensional (3D) image volume of a patient prostate, wherein said first 3D image volume is an magnetic resonance imaging (MRI) image and wherein said first 3D image volume is disposed within a first frame of reference;
identifying a first boundary surface of said first 3D image volume;
obtaining, at said processing platform, a substantially real-time second 3D image volume of the patient prostate from an ultrasound device, wherein said second 3D image volume is disposed in a second frame of reference;
identifying a second boundary surface of said second 3D image volume;
operating said processor to register said first and second boundary surfaces of said first and second 3D image volumes, respectively, to generate a surface transformation between said boundary surfaces; and
applying said surface transformation to said one of said 3D image volumes to generate a field transformation between said first and second 3D image volumes.

2. The method of claim 1, further comprising:
applying said field transformation to said second 3D image volume, wherein said substantially real-time second 3D image volume is displayed the first frame of reference of said pre-acquired first 3D image volume.

3. The method of claim 2, further comprising:
identifying a point of interest within said first 3D image volume;
applying said field transformation to said point of interest, wherein said point of interest is transformed into said second frame of reference of said substantially real-time second 3D image volume.

4. The method of claim 3, further comprising:
displaying said point of interest in said substantially real-time second 3D image volume.

5. The method of claim 1, wherein said pre-acquired first 3D image volume further comprises:
at least one region of interest (ROI) delineated within said 3D volume, wherein coordinates of a geometric definition of said ROI are saved in the first frame of reference.

6. The method of claim 5, further comprising:
applying said field transformation to said geometric definition of said at least one ROI in said first frame of reference to generate a corresponding at least one ROI in said second frame of reference.

7. The method of claim 1, wherein identifying a boundary surface for at least one of said first and second 3D image volumes comprises:
segmenting a boundary of said prostate.

8. The method of claim 1, wherein identifying a boundary surface for at least one of said first and second 3D image volumes comprises:
generating a mesh surface including a plurality of vertices and facets.

9. The method of claim 8, wherein said surface transformation comprises a set of vectors extending between corresponding vertices of a first mesh surface corresponding to said pre-acquired first 3D image volume and a second mesh surface corresponding to said second 3D image volume.

10. The method of claim 1, further comprising:
prior to registering said first and second boundary surfaces, rigidly aligning said first and second boundary surfaces to a substantially common frame of reference.

11. The method of claim 1, further comprising:
applying said field transformation to said second 3D image volume, wherein said substantially real-time second 3D image volume is transformed into the first frame of reference of said pre-acquired first 3D image volume;
blending a portion of each corresponding voxel of said first and second 3D image volumes to generate a blended image disposed in said first frame of reference.

12. The method of claim 11, further comprising:
selectively adjusting the blending factor of said composite image to vary the composition of said composite image.

13. The method of claim 1, further comprising:
generating a guidance output for guiding an instrument to a physical location corresponding with the location within said prostate as represented by said second 3D image volume.

14. A method for use in prostate treatment procedures where a pre-procedure Magnetic Resonance Imaging (MRI) image is utilized in conjunction with a current ultrasound image to guide a medical procedure, comprising:
obtaining, at a processing platform, a substantially real-time ultrasound image of a patient prostate;
using said processing platform, transforming said real-time ultrasound image into a frame of reference of a previously acquired MRI image of said patient prostate to compute a transformation between said ultrasound image and said MRI image;
identifying at least one region of interest (ROI) in said previously acquired MRI image;
applying said transformation to said at least one ROI using said processing platform, wherein said ROI is transformed into a frame of reference of said real-time image to generate a real-time ROI;
generating an display of said real-time ROI in said real-time image of said prostate.

15. The method of claim 14, further comprising:
generating a guidance output for guiding an instrument to a physical location corresponding with the location of said real-time ROI in said real-time image of said prostate.

16. The method of claim 14, wherein transforming said real-time image generates a registered ultrasound image, wherein said registered ultrasound image is disposed in the frame of reference of said previously acquired MRI image.

17. The method of claim 14, further comprising:
blending an intensity of each corresponding voxel of said registered ultrasound image and said previously acquired MRI image to generate a blended image, wherein said blended image is displayed.

18. The method of claim 17, further comprising:
selectively adjusting a blending proportion of said MRI image and said registered ultrasound image of said composite image to vary the composition of said composite image.

19. The method of claim 17, wherein identifying said at least one ROI comprises using said composite image to identify said at least one ROI.

20. The method of claim 14, wherein identifying said at least one ROI comprises identifying at least set one predetermined coordinates associated with at least one pre-identified ROI.
